(19) United States
(12) Patent Application Publication
(10) Pub. No.: US 2012/0207838 A1
(43) Pub. Date: Aug. 16, 2012

(54) Treatment of Psoriasis Using Oral Dosage Forms of Nitrone Spin Traps

(76) Inventor: Nicholas V. Perricone, Meriden, CT (US)

(21) Appl. No.: 13/024,478

(22) Filed: Feb. 10, 2011

(51) Int. Cl. A61K 31/15; A61K 9/16; A61P 17/06; A61K 9/00

(52) U.S. Cl. 424/489; 514/640; 424/400

(57) ABSTRACT

Psoriasis is treated by oral administration of a pharmaceutical composition containing a nitrone spin trap such as α-phenyl t-butyl nitrone (PBN) and derivatives thereof. Preferred compositions and method of treatments further comprise at least one adjunct ingredient including fatty acid esters of ascorbic acid such as ascorbyl palmitate and ascorbyl stearate. The pharmaceutical composition can be prepared as an immediate release formulation or controlled released formulations.

### FIELD OF THE INVENTION

The present invention relates to the skin disease known as psoriasis and, more particularly, to pharmaceutical compositions comprising nitrone spin trap and methods of use thereof for the treatment of psoriasis.

### BACKGROUND OF THE INVENTION

Psoriasis is a lifelong skin disease that occurs when faulty signals in the immune system cause skin cells to regenerate too quickly, on the order of every three to four days instead of the usual 30-day cycle. Extra skin cells build up on the skin's surface, forming red, flaky, scaly lesions that can itch, crack, bleed and be extremely painful. Psoriasis generally involves the joints, limbs and scalp but it can appear anywhere on the body, covering some people from head to toe. More than 7 million Americans have been diagnosed with psoriasis and/or psoriatic arthritis, a degenerative disease of the joints and connective tissues associated with psoriasis. Psoriasis typically first strikes people between the ages of 15 and 35, but can affect anyone at any age, including children.

Psoriasis is characterized by erythematous eruptions, often in papules or plaques, and usually having a white, silvery scale. Psoriasis is generally considered an inflammatory skin condition. Other inflammatory skin conditions include atopic dermatitis (eczema), seborrhoeic dermatitis, rosacea, acne, as well as contact dermatitis (typically arising from allergic reaction to poison ivy and other allergens).

Psoriasis is persistent and unpredictable in its course. The exact etiology of psoriasis is unknown. It is postulated that psoriasis may involve abnormalities in essential fatty acid metabolism, free radical generation, lipid peroxidation, and/or release of lymphokines. One study showed that lipid peroxidation mediated by free radicals is one of the important causes of cell membrane destruction and cell damage associated with psoriasis. Tekin N. S., Accumulation of oxidized low-density lipoprotein in psoriatic skin and changes of plasma lipid levels in psoriatic patients, Mediators Inflamm. 12, 78454 (2007). Other studies also reported that oxidative stress and increased free-radical generation link to psoriasis. Rashmi R, Clin. Exp. Dermatol. 34, 658-63 (2009); Cooke, M. S., Oxidative DNA damage: mechanisms, mutation, and disease, the FASEB Journal, 17, 1195-1214 (2003).

Despite a voluminous scientific literature and numerous treatment strategies, there is still no effective treatment for psoriasis that is completely without side effects. Conventional therapeutic regimens for psoriasis include topical or intralesional application of corticosteroids, anthralin, tazarotene (a retinoid), acitretin (a second-generation retinoid), calcipotriene (vitamin D3) and/or zinc compounds, and/or selenium compounds, and/or coal tar compounds; or various light therapies; or an oral or injected systemic agent. No single therapy is ideal, and it is rare for a patient not to be treated with several alternatives during the relapsing and remitting course of the disease. Whereas conventional systematic treatment can induce prompt resolution of psoriatic lesions, suppression often requires ever-increasing doses, sometimes with toxic side effects, and tapering of therapy may result in rebound phenomena with extensions of lesions, possibly to exfoliation. Other inflammatory skin conditions are typically treated with the same types of therapies. Thus, there is a continuous effort in finding an effective and safe drug for treating chronic psoriasis and other inflammatory skin conditions.

Nitrone spin traps are potent free radical scavengers and antioxidants, and are commonly used as analytical tools to study free radicals. Nitrone behave as spin trapping agents when a diamagnetic nitrone compound (the “spin trap”) reacts with a transient free radical species (having the “spin”) to provide a relatively more stable radical species (referred to as the “spin adduct”). The spin adduct may be detectable by electron paramagnetic resonance (EPR) spectroscopy, or electron spin resonance (ESR) if the spin adduct has a reasonable lifetime. Thus, spin trapping allows previously unobservable free radicals to be identified and studied using ESR, EPR, and related techniques. Sudha Rana, Electron paramagnetic resonance spectroscopy in radiation research: Current status and perspectives, J. Pharmacy & BioAllied Sciences, 2, 80-87 (2010).

The use of nitrones as spin traps for studying unstable free radicals has been applied to biological systems. In this regard, α-phenyl t-butyl nitrone (PBN), 5,5-dimethyl pyrroline N-oxide (DMPO) and related compounds have been used to identify superoxide (O₂.) and hydroxyl radicals (HO.) in biological systems. Additionally, such nitrones have been used to study lipid peroxidation and other free radical-induced biological processes.

Besides serving as research aids or diagnostic tools, nitrone spin traps have been used in connection with therapeutic applications. For example, PBN and derivatives thereof, have been reported for the treatment of a wide variety of disease conditions arising from or characterized by free radical-induced oxidative damage. Such disease conditions include, for example, disorders of the central nervous system (CNS) and the peripheral nervous system, such as stroke, Parkinsonism, traumatic nerve damage and the like, and disorders of the peripheral organs, such as atherosclerosis, cardiac infarction, ulcerative colitis and the like. Nitrone spin traps have also been reported to treat arthritis.

### SUMMARY OF THE INVENTION

The primary object of this invention is to provide prevention and treatment methods for psoriasis, and more particularly, to provide a safe therapeutic agent in oral dosage form to a person in need.

This and other objectives of the invention are accomplished by the present invention, which provides a pharmaceutical composition containing a nitrone spin trap, preferably PBN and derivatives, which is formulated suitable for oral administration, for the treatment and prevention of psoriasis.

In order to provide an effective therapeutic agent for treating psoriasis, it is desirable to be able to administer the nitrone spin traps at high doses, especially initially. Thus, the nitrone spin traps used to treat psoriasis conditions should be non-toxic or have very low toxicity.

It is also important that the nitrone spin traps can efficiently reach the biological site where the free radicals are generated so that the radicals are trapped by the nitrone spin traps before the damages occur. Thus, it is particularly desirable to be able to optimize the bioavailability and cell permeability of the nitrone spin traps.

It is further desirable that the nitrone spin traps are administered no more than two times a day to improve patient compliance.

Accordingly, the present invention provides nitrone spin traps having low toxicity, increased bioavailability and cell permeability, and less frequent administrations for effective oral treatment of psoriasis.

The amount of the nitrone spin trap necessary to bring about the therapeutic treatment of psoriasis is not fixed per se, and necessarily is dependent upon the severity and extent of the disease, the form of the nitrone spin trap employed, and the concentration of the nitrone spin trap in the pharmaceutical composition. A typical daily dosage ranges from 0.1 to 100 mg/kg/day, preferably from 10 to 60 mg/kg/day, and more preferably from 15 to 45 mg/kg/day. It is beneficial to take oral dosages, in equally divided portions, at predetermined intervals, ranges from one time to five times a day, preferably two to three times a day.

The pharmaceutical composition can be in various oral dosage forms such as tablets, sublingual tablets, capsules, powders, granules or fine granules, or suspensions in a non-aqueous liquid such as syrups, emulsions or draft that contain a prescribed amount of the nitrone spin traps. The prescribed amount ranges from about 50 mg to about 800 mg, preferably 100 mg to 400 mg and more preferably about 250 mg per unit.

The pharmaceutical composition may further comprise at least one adjunct ingredient such as fatty acids, and fatty acid esters of ascorbic acid. The amount of each adjunct ingredient is at a range of about 0.025 w/w % to about 0.5 w/w %.

The pharmaceutical composition can be prepared as a conventional immediate release formulation or controlled released formulations. The controlled released formulations include, but not limited to, the ones employ enteric coatings, solid dispersion, a combination thereof, and multilayer structured techniques. A preferred agent for the solid dispersion is polyphosphatidylcholine which greatly improve and prolong the antioxidant activities of the nitrone spin trap.

### DETAILED DESCRIPTION OF THE INVENTION

Pharmaceutical compositions containing nitrone spin trap according to the present invention are orally administrated to a person in need for the prevention and treatment of psoriasis and other skin inflammatory diseases. Since psoriasis can be a chronic disease, oral administration is preferred for the convenience and tolerance of patients. Oral administration also has the advantage of effectively preventing psoriasis before its eruption, while other administration means such as topical administration usually can only be applied after the eruption of psoriasis.

As used herein, the term “nitrone spin trap(s)” used herein and after refers to both nitrone spin traps and derivatives thereof.

Any nitrone spin traps, either in straight chain or in cyclic configuration, may be employed in compositions of the invention. Common nitrone spin traps can typically be purchased from Sigma-Aldrich Chemical Co. or other chemical vendors. Uncommon nitrone spin traps, for example, azulenyl nitrone spin traps and furan nitrone spin traps, can be synthesized following the procedures known in the art. U.S. Pat. No. 6,376,540 to Kelleher and U.S. Pat. App. No. 20080167474 to Becker have reported the synthesis of these types of nitrone spin traps, the disclosure of which is incorporated by references in entirety. Regardless the source of the spin traps, it is important that the spin traps are sufficiently pure, ideally with greater than 98% purity, and any impurities should be inert in the sense of not bringing about a deactivation of the nitrone spin traps. Preferred spin traps should have minimal or no toxicity to normal cells. Suitable nitrone spin traps include, but is not limited to, phenyl N-tert-butylnitrone, also referred to as α-phenyl t-butyl nitrone (PBN), 5,5-dimethyl pyrroline N-oxide (DMPO), α-(4-pyridyl 1-oxide)-N-tert-butylnitrone (POBN), 3,3,5,5-tetramethyl-l-pyrroline N-oxide, and 2,4,4,6-tri-tert-butylnitrosobenzene (BNB). Other nitrone spin traps described in U.S. Pat. Nos. 5,405,874, 5,681,845, 5,681,965, 6,002,001 and RE. 36,594 can also be used, the disclosure of which is incorporated herein by reference.

The most preferred nitrone spin traps are PBN and derivatives thereof, because they have no measurable effect on normal or uninjured cells. PBN and derivatives thereof have the following general formula:

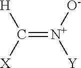

wherein X is phenyl or substituted phenyl with up to five substitutions on the phenyl ring, and each substitution is independently (can vary within the molecule) selected from the group consisting of hydrogen, halogen, alkyl, substituted alkyl, alkaryl, alkoxyl, alkenyl, and amino; and Y is selected from the group consisting of alkyl, substituted alkyl, alkenyl, alkynyl, naphthyl, heterocyclic, alkcycloalkyl, cycloalkyl and cycloalkenyl.

“Alkyl” refers to monovalent alkyl groups preferably having from 1 to about 12 carbon atoms, more preferably 1 to 8 carbon atoms and still more preferably 1 to 6 carbon atoms. This term is exemplified by groups such as methyl, ethyl, n-propyl, isopropyl, n-butyl, isobutyl, tert-butyl, n-hexyl, n-octyl, tert-octyl and the like.

“Amino” refers to primary, secondary and tertiary alkyl substituted amino groups and the like.

“Substituted alkyl” refers to an alkyl group preferably having from 1 to about 12 carbon atoms, more preferably 1 to 8 carbon atoms and still more preferably 1 to 6 carbon atoms, which is substituted with from 1 to 3 substituents selected from the group consisting of alkoxy, amino, mono- and dialkylamino, aminoacyl, aminocarbonyl, alkoxycarbonyl, aryl, carboxyl, cyano, halo, heterocyclic, hydroxy, nitro, thioalkoxy and the like. A preferred substituted alkyl group is the trifluoromethyl group.

“Alkaryl” refers to alkylene-aryl groups preferably having from 1 to 10 carbon atoms in the alkylene moiety and from 6 to 14 carbon atoms in the aryl moiety. Such alkaryl groups are exemplified by benzyl, phenethyl, and the like.

“Alkcycloalkyl” refers to -alkylene-cycloalkyl groups preferably having from 1 to 10 carbon atoms in the alkylene moiety and from 3 to 8 carbon atoms in the cycloalkyl moiety. Such alkcycloalkyl groups are exemplified by cyclopropyl, cyclopentyl, cyclohexyl, and the like.

“Alkoxy” refers to the group “alkyl-O—”. Preferred alkoxy groups include, by way of example, methoxy, ethoxy, n-propoxy, isopropoxy, n-butoxy, tert-butoxy, sec-butoxy, n-pentoxy, n-hexoxy, 1,2-dimethylbutoxy, and the like.

“Alkenyl” refers to alkenyl groups preferably having from 2 to 10 carbon atoms and more preferably 2 to 6 carbon atoms and having at least 1 and preferably from 1-2 sites of alkenyl unsaturation. Preferred alkenyl groups include ethenyl, n-propenyl, isopropenyl, and the like.

“Alkynyl” refers to alkynyl groups preferably having from 2 to 10 carbon atoms and more preferably 2 to 6 carbon atoms and having at least 1 and preferably from 1-2 sites of alkynyl unsaturation. Preferred alkynyl groups include ethynyl, propargyl, and the like.

“Cycloalkyl” refers to cyclic alkyl groups of from 3 to 10 carbon atoms having a single cyclic ring or multiple condensed rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantanyl, and the like.

“Cycloalkenyl” refers to cyclic alkenyl groups of from 4 to 10 carbon atoms having a single cyclic ring and at least one point of internal unsaturation which can be optionally substituted with from 1 to 3 alkyl groups. Examples of suitable cycloalkenyl groups include, for instance, cyclopent-3-enyl, cyclohex-2-enyl, cyclooct-3-enyl and the like.

“Halo” or “halogen” refers to fluoro, chloro, bromo and iodo. Preferred halo groups are either fluoro or chloro.

“Heterocycle” or “heterocyclic” refers to a monovalent saturated or unsaturated group having a single ring or multiple condensed rings, from 1 to 10 carbon atoms and from 1 to 4 hetero atoms selected from nitrogen, sulfur or oxygen within the ring. Examples of heterocycles include, but are not limited to, morpholine, piperazine, imidazolidine, pyrrolidine, piperidine and the like.

“Naphthyl” refers to naphthyl ring and can optionally be substituted with from 1 to 3 substituents selected from the group consisting of alkyl, substituted alkyl, alkoxy, alkenyl, alkynyl, amino, aminoacyl, aminocarbonyl, alkoxycarbonyl, aryl, carboxyl, cyano, halo, hydroxy, nitro, trihalomethyl and the like.

The daily dosage of the nitrone spin traps may vary with the administration route, the symptom to be treated, and the patient condition. An appropriate dosage for treating psoriasis ranges from 0.1 to 100 mg/kg/day, preferably from 10 to 60 mg/kg/day, and more preferably from 15 to 45 mg/kg/day. It is beneficial to take oral dosages, in equally divided portions, at predetermined intervals, ranges from one time to five times a day, preferably two to three times a day. It is noticed that less frequent doses per day generates better patient compliance. Repetitive daily administration may be desirable and will vary according to the conditions outlined above. It is usually contemplated that the present invention delivers nitrone spin trap at a relatively high dosage during treatment and gradually titration down to a low concentration maintenance dosage. It generally is the case that gradual improvement is noted with each successive application.

The suitable dosage forms of the present invention include, but not limited to, tablets, sublingual tablets, capsules, powders, granules or fine granules, or suspensions in a non-aqueous liquid such as syrups, emulsions or draft (pro re nata preparation) that contain prescribed amount of the active ingredient. The prescribed amount may be in a range of about 50 mg to about 800 mg, preferably 100 mg to 400 mg and more preferably about 250 mg per unit.

Nitrone spin traps may be administered alone or in combination with pharmaceutical formulations containing other active ingredients suitable for psoriasis under treatment to facilitate control of the dosage.

Some embodiments of this invention contain at least one other adjunct ingredient in addition to the nitrone spin trap. Adjunct ingredients include, but are not limited to, fatty acids, which may be in the form of fatty acid esters. Many embodiments employ more than one adjunct ingredient.

As used herein, the term “fatty acid” has reference to and encompasses the all isomers of the free acid and structurally related, biologically equivalent derivatives such as salts and esters. Suitable fatty acids include, but not limited to, long chain fatty acids such as lipoic acid and ascorbic acid, essential fatty acids, such as omega-3 fatty acid, linoleic acid, and omega-6 fatty acid, arachidonic acid.

The most preferred fatty acid is ascorbic acid (vitamin C), which is often employed in the form of fat-soluble fatty acid esters of ascorbic acid. The more oxidation-resistant saturated fatty acid esters of ascorbic acid are preferred, including, but not limited to, ascorbyl laurate, ascorbyl myristate, ascorbyl palmitate, ascorbyl stearate, and ascorbyl behenate. Ascorbyl palmitate is used in one embodiment. As denoted herein, where fatty acid esters are described, e.g., ascorbyl stearate, compositions having predominantly that ester, e.g., predominantly ascorbyl stearate, are included. The esters may be prepared using hydrogenated oils or fats, or fractions thereof, and contain small amounts of another ester. Ascorbyl stearate prepared using canola, for example, commonly contain about 4% ascorbyl palmitate. It is an advantage of the invention that where fatty acid esters of ascorbic acid are employed as an adjunct ingredient, they help stabilize and solubilize the nitrone spin trap in the pharmaceutical composition.

Where adjunct ingredients are employed, the amount of adjunct ingredients necessary to bring about enhanced prevention and/or therapeutic skin treatment in conjunction with the nitrone spin trap is not fixed per se, and necessarily is dependent upon the identity and form of the adjunct ingredients employed, the concentration of the adjunct ingredients when employed with a carrier, the user's skin type, and, where present, the severity and extent of the patient's pathological skin condition. Since PBN degrades at pH less than approximately 3 to 4, it is important that the amount of the fatty acid or other ingredients added would not bring the pharmaceutical composition to a pH below 4. Many embodiments contain from about 0.025% to 0.5% of the fatty acid or esters thereof.

Pharmaceutical compositions of the present invention are prepared by formulating nitrone spin traps, and adjunct ingredients if any, with carriers into dosage forms suitable for oral administration. Optionally one or more auxiliary ingredients selected from buffers, flavors, surfactants, viscosants, lubricants, etc. can also be added in the formulation.

A suitable carrier should be one in which nitrone spin traps is soluble per se or is effectively solubilized. Where employed, the carrier is inert in the sense of not bringing about a deactivation or oxidation of the nitrone spin traps, and in the sense of not bringing about any adverse effect on the skin areas to which it is applied. The carriers used for conventional immediate release pharmaceutical formulations include excipients, binders, disintegrators, etc., depending on the dosage form chosen. Typical examples of excipients include starch, lactose, sucrose, glucose, mannitol and cellulose, and examples of binders include polyvinylpyrrolidone, starch, sucrose, hydroxypropyl cellulose, Arabic gum. Examples of disintegrators include starch, agar, gelatin powder, cellulose, CMC. Other materials as well as processing techniques and the like are set forth in Remington's Pharmaceutical Sciences, 17^{th} edition, 1985, Mack Publishing Company, Easton, Pa., which are incorporated by reference.

The present invention can also be prepared in a controlled release formulation. Controlled release within the scope of the invention can be taken to mean any one of a number of extended release dosage forms. A preferred controlled release formulation releases the active ingredient gradually over time at a controlled rate of release over 4 hours or more so that a desired level of nitrone spin traps is maintained in blood serum to provide long term therapeutic effect and also to avoid toxicity that may be associated with the spike of the drug concentration of the immediate release formulations. A controlled release formulation means that patients may take the nitrone spin traps in a larger dose at less frequency to reach same therapeutic effect. This feature is particularly beneficial to a chronic disease such as psoriasis because it helps to improve patient compliance. There are corporations with specific expertise in drug delivery technologies including controlled release oral formulations such as Alza corporation and Elan. Numerous patents disclose the controlled release oral formulations, such as U.S. Pat. Nos. 5,637,320, 5,505,962, 5,641,515, 7,118,762, 7,338,667, and 7,572,462, the teachings of which are incorporated by references in entirety.

One preferred type of oral controlled release formulation utilizes enteric coating as a carrier. Enteric coating allows the active ingredients to remain physically incorporated in the dosage form for a specific period after oral ingestion and be released from the coatings when the coatings are dissolved in the digestive system at specific pH. The desired pH for the coating dissolution is above pH 5.5 which present in the small intestine. Suitable enteric coating agents include, but not limited to, hydroxypropylmethylcellulose phthalate, methacryclic acid-methacrylic acid ester copolymer, polyvinyl acetate-phthalate and cellulose acetate phthalate.

Another preferred type of oral controlled release formulation employs solid dispersions. Solid dispersions may be defined as a dispersion of active ingredients in an inert carrier or matrix in the solid state. Akihiko Hasegawa, Super Saturation Mechanism of Drugs from Solid Dispersions with Enteric Coating Agents, Chem. Pharm. Bull. 36: 4941 4950 (1998). Solid dispersions may be used to improve the solubilities and/or dissolution rates of poorly soluble active ingredients such as fatty nitrones. Hiroshi Yuasa, et al., Application of the Solid Dispersion Method to the Controlled Release Medicine. III. Control of the Release Rate of Slightly Water-Soluble Medicine From Solid Dispersion Granules, Chem. Pharm. Bull. 41:397 399 (1993).

The inert carrier may have an influence on the dissolution characteristics of the dispersed drug because the dissolution rate of the drug from a surface may be affected by the carrier in the solid dispersion formulation. For example, a water-soluble carrier may result in a fast release of the nitrone spin trap from the matrix, or a poorly soluble or insoluble carrier may lead to a slower release of the nitrone spin trap from the matrix. The solubility of the nitrone spin trap may also be increased owing to some interaction with the carriers.

Phosphatidylcholine (PC) is a known carrier for solid dispersions. PC is an amphoteric but water-insoluble lipid, which may improve the solubility of otherwise insoluble nitrones, such as azulenyl nitrones, in an amorphous state in phosphatidylcholine solid dispersions. Makiko Fujii, et al., Dissolution of Bioavailibility of Phenyloin in Solid Dispersion with Phosphatidylcholine, Chem. Pharm. Bull 36:4908 4913 (1988). Moreover, because PC itself is a major constituent of cell membranes, PC readily penetrates in cell membranes. Thus, when employed, PC greatly enhances the antioxidant activity of the composition because it facilitates the nitrone spin traps to disperse into cell membranes of psoriatic lesions in quantities sufficient to reach therapeutic levels.

A particular preferred PC carrier in accordance with the present invention is polyenylphosphatidylcholine (PPC). By “polyenylphosphatidylcholine” it meant any phosphatidylcholine (PC) bearing two fatty acid substituents, wherein at least one is an unsaturated fatty acid with at least two double bonds. It is discovered that PPC greatly improves and prolongs the antioxidant activities of the nitrone spin trap of the solid dispersion. Preferred polyenylphosphatidylcholines contain at least one linoleic (18:2) group, most preferably two, in a cis geometrical configuration typical of natural products, which presents in the preparation at levels of at least about 25%, preferably at least about 40% by weight. Other forms of PPC can also be used as those set out in U.S. Pat. No. 6,797,459 at column 3 lines 34 to 52. Without wishing to be bound by theory, it is believe that the superior antioxidant effect is attributed to the active antioxidizing nature of PPC, and thus PCC assists in protecting against lipid peroxidation and liver damage, including fibrosis and cirrhosis. Aleynik, S. I., et al., J. Investig. Med. 47: 507-512 (1999).

A third preferred type of oral controlled release formulation has the nitrone spin traps included in a solid dispersion system and then being coated with an enteric polymer.

A fourth preferred type of oral controlled release formulation uses a multi-layers, usually double layers dosage form. For example, the first extragranular layer may be formulated as an immediate release granulate, and the second intragranular layer may be formulated in a controlled manner, incorporating enteric coatings, solid dispersion, or the combinations thereof. This formulation provides a wide range of desirable effects in that a high dose of the nitrones can be immediately released to accommodate most severe psoriasis conditions followed by a low dose of the nitrones for continued treatment and maintenance.

The mechanisms of action of the therapeutic effectiveness of the nitrone spin traps for psoriasis are not fully understood at this time. One theory is that PBN and derivatives reduce levels of the free radicals, especially hydroxyl and superoxide radicals that implicate psoriasis, by forming stable complexes with the free radicals, thus inhibit the lipoxygenase oxidation pathway and interrupt the inflammatory cascade processes which result in the regulation of the cell growth cycle. Accordingly, skin cells are produced in a normal manner instead of the accelerated and damaged state typical of psoriasis and other inflammatory skin conditions.

This theory can be supported, in part, by the fact that the nitrone spin traps, such as PBN and derivatives, have little or no measurable effect on normal cells. A rational explanation is that stable complexes are formed between free radicals and nitrone spin traps during the treatment. Because nitro spin traps can only react with the free radicals produced by those damaged cells, normal cells with no free radicals could not be affected by nitrone spin traps.

However, the high efficiency of the present invention in the treatment of psoriasis suggests that PBN and derivatives may function beyond their antioxidant capabilities. Without wishing being bound by theory, it is believed that PBN and derivatives act as Michael acceptor pharmacophores in binding to, and thus inactivating, the transcription factors which contribute to the pathogenesis of psoriasis. This mechanism of action is proposed based on the chemical structure of PBN and derivatives which have a chemical structure in which an active nitrogen atom is adjacent an oxygen atom, so that the carbon atom next to the nitrogen becomes electron deficient. This allows PBN and derivatives to act as electrophilic Michael acceptors to bind with the cysteine residues on many different enzyme genera and transcription factors. Since the Michael reaction is irreversible, PBN and derivatives thus permanently inhibit the cellular signal transduction pathways that lead to psoriatic lesions.

It has recently been proposed that a positive feedback loop is involved in psoriatic lesions that strengthens the effect of Reactive Oxygen Species (ROS) and amplifies the production of proinflammatory cytokines: (1) ROS trigger activation of MAPK/AP-1, NF-κB, and JAK-STAT signaling pathways, which subsequently induce iNOS, and (2) ONOO— is generated by the reaction of NO and oxidant O2.—, resulting in the activation of NF-κB and AP-1, which evoke the expression of the target genes. Qiang Zhou, Oxidative stress in the pathogensis of psoriasis, Free Radical Biology & Medicine, 47: 891-905 (2009).

PBN and derivatives should bind to and inactivate NF-kB, a molecule which is significantly expressed in psoriatic skin and which up-regulates over one hundred proinflammatory compounds including those implicated in the development of psoriatic lesions. Moreover, PBN and derivatives should bind to and inactivate transcription factor AP-1. As a consequence, the effect of PBN and derivatives should be to inhibit the primary cellular signal transduction pathways that lead to psoriatic lesions and therefore provide for prevention and treatment of psoriasis.

In addition, PBN and derivatives should also activate Nrf2, a transcription factor which up regulates about twenty different cyto protective enzymes, phase 2 proteins and antioxidant enzymes, thus inhibiting NO production and providing a secondary pathway for prevention and treatment of psoriasis.

The above described mechanisms of action mean that relatively small amounts of PBN and derivatives are sufficient for effective prevention and treatment of psoriasis. By inactivating the key transcription factors at an early stage of the pathogenesis pathways, PBN and derivatives block the signal transduction pathways, and prevent the subsequent cascading, catalytic, and up-regulated expression of proinflammatory cytokines and chemokines in the psoriasis stage. The high efficacy of PBN and derivativesis a particularly beneficial aspect of the present invention.

Methods and compositions of the present invention are also useful for treating other type of inflammatory skin conditions, such as dermatitis, rosacea, seborrhea, eczema, xerosis (dry skin), thermal and radiation burns. It is an advantage of the invention that oral administration of nitrone spin traps provides a simple, nontoxic, and effective method for treating all kinds of skin damages, including psoriasis. It is another advantage of the invention that nitrone spin trap is particularly efficacious in the treatment of certain skin conditions that do not respond to topical corticosteroids. It is a further advantage that nitrone spin trap can be formulated in various oral administrable dosage forms that reduce the frequency of drug intake and improve patient compliance.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims.

What is claimed is:

1. A method for the prevention and treatment of psoriasis, comprising administrating to a patient in need an oral dosage form of a pharmaceutical composition comprsing an effective amount of a nitrone spin trap having the formula (1): 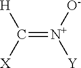
wherein
X is phenyl or substituted phenyl with up to five substitution groups on the phenyl ring, wherein each said substitution groups is independently (can vary within the molecule) selected from the group consisting of hydrogen, halogen, alkyl, substituted alkyl, alkaryl, alkoxyl, alkenyl, and amino, and
Y is selected from the group consisting of alkyl, substituted alkyl, alkenyl, alkynyl, naphthyl, heterocyclic, alkcycloalkyl, cycloalkyl and cycloalkenyl.

2. The method according to claim 1, wherein said nitrone spin trap is α-phenyl-tert-butylnitrone.

3. The method according to claim 1, wherein said effective amount is 0.1 to 100 mg/kg/day.

4. The method according to claim 3, wherein said effective amount is 10 to 60 mg/kg/day.

5. The method according to claim 4, wherein said effective amount is 15 to 45 mg/kg/day.

6. The method according to claim 1, wherein said oral dosage form is selected from the group consisting of tablets, sublingual tablets, capsules, powders, granules, and suspensions.

7. The method according to claim 6, wherein said oral dosage form contains a prescribed amount of said nitrone spin trap in the range from about 50 mg to about 800 mg per unit.

8. The method according to claim 7, wherein said prescribed amount is in the range from about 100 mg to about 400 mg per unit.

9. The method according to claim 8, wherein said prescribed amount is about 250 mg per unit.

10. The method according to claim 1, wherein said pharmaceutical composition further comprises an adjunct ingredient selected from the group consisting of fatty acid, fatty acid ester of ascorbic acid, and the mixture thereof.

11. The method according to claim 10, wherein said adjunct ingredient is from about 0.025% to about 0.5% by weight of the composition.

12. The method according to claim 10, wherein said fatty acid is selected from the group consisting of lipoic acid, ascorbic acid, linoleic acid and arachidonic acid.

13. The method according to claim 10, wherein said fatty acid ester of ascorbic acid is selected from the group consisting of ascorbyl laurate, ascorbyl myristate, ascorbyl palmitate, ascorbyl stearate, and ascorbyl behenate.

14. The method according to claim 13, wherein said fatty acid ester of ascorbic acid is selected from the group consisting of ascorbyl palmitate, ascorbyl stearate, and the mixture thereof.

15. The method according to claim 1, wherein said pharmaceutical composition further comprises an oral carrier.

16. The method according to claim 15, wherein said oral carrier is an immediate release drug carrier.

17. The method according to claim 15, wherein said oral carrier is a controlled release drug carrier.

18. The method according to claim 17, wherein said controlled release drug carrier is enteric coating composed by an enteric coating agent.

19. The method according to claim 18, wherein said enteric coating agent is selected from the group consisting of hydroxypropylmethylcellulose phthalate, methacryclic acid-methacrylic acid ester copolymer, polyvinyl acetate-phthalate and cellulose acetate phthalate.

20. The method according to claim 19, wherein said controlled release drug carrier is a solid dispersion carrier.

21. The method according to claim 20, wherein said solid dispersion carrier is polyenylphosphatidylcholine.

22. The method according to claim 21, wherein dilinoleoylphosphatidylcholine is the most abundant phosphatidylcholine species in said polyenylphosphatidylcholine.

23. The method according to claim 22, wherein dilinoleoylphosphatidylcholine comprises at least about 25% by weight of said polyenylphosphatidylcholine.

24. The method according to claim 23, wherein dilinoleoylphosphatidylcholine comprises at least about 40% by weight of said polyenylphosphatidylcholine.

25. The method according to claim 19, wherein said controlled release drug carrier is a solid dispersion system being coated with an enteric polymer.

26. The method according to claim 19, wherein said controlled release drug carrier is a double layers dosage carrier in which the first extragranular layer is an immediate release granulate, and the second intragranular layer is a controlled release granulate.

27. A composition for the prevention and treatment of psoriasis, comprising:
a nitrone spin trap having the chemical structure of 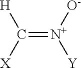
wherein:
X is phenyl or substituted phenyl with up to five substitution groups on the phenyl ring, wherein each said substitution groups is independently (can vary within the molecule) selected from the group consisting of hydrogen, halogen, alkyl, substituted alkyl, alkaryl, alkoxyl, alkenyl, and amino, and
Y is selected from the group consisting of alkyl, substituted alkyl, alkenyl, alkynyl, naphthyl, heterocyclic, alkcycloalkyl, cycloalkyl and cycloalkenyl.
a fatty acid ester, and
an oral carrier.

28. The composition according to claim 27, wherein said nitrone spin trap is α-phenyl-tert-butylnitrone.

29. The composition according to claim 27, wherein said oral carrier is polyenylphosphatidylcholine.
